(19) United States
(12) Patent Application Publication
(10) Pub. No.: US 2003/0088266 A1
(43) Pub. Date: May 8, 2003

(54) Method of fusing electroprocessed matrices to a substrate

(76) Inventor: Gary L. Bowlin, Mechanicsville, VA (US)

Correspondence Address: John H. Thomas, P.C., 1561 East Main Street, Richmond, VA, 23219, US

(21) Appl. No.: 10/282,378

(22) Filed: Oct. 29, 2002

Related U.S. Application Data
(60) Provisional application No. 60/330,890, filed on Nov. 2, 2001.

(51) Int. Cl. A61M 29/00

(52) U.S. Cl. 606/200

(57) ABSTRACT

A medical device for filtering fluid passing through a lumen in a body includes a flexible frame and an electrospun matrix. The flexible frame includes a plurality of wires intersecting to define a perimeter of an open space. The electrospun matrix including a multiplicity of fibers that is fused to the frame and extends across the open space to define a multiplicity of pores.

This application claims the benefit of U.S. Provisional Application No. 60/330,890 filed Nov. 2, 2001.

The present invention relates to a method of electroprocessing a polymer onto a target substrate, and specifically to the further processing steps that prevent the delamination of the polymer matrix from the target substrate. Fusion of the matrix onto the substrate enhances the attachment of the matrix to the substrate and reduces or eliminates the likelihood of delamination.

### BACKGROUND OF THE INVENTION

Electroprocessing may be used to form a matrix coating of polymer onto a substrate. There are many potential uses of an electroprocessed coating including biomedical applications. For instance, it is possible to coat devices or implants in order to obtain favorable surface characteristics. In one particular application, fibers may be electrospun onto a filter. A specific embodiment is described in detail in United States Patent Application Serial No. 10/056,588 (Publication No. US2002/0128680 A1, published Sep. 12, 2002), entitled “Distal Protection Device With Electrospun Polymer Fiber Matrix”. This reference is incorporated by reference herein. The filter substrate may be any type of material, but it is commonly metallic. The filter is typically a fine metal mesh. In the embodiment noted, the filter is a distal protection device having a metal mesh substrate. By layering electrospun fibers onto the wire mesh, the pore size or other performance attributes of the filter may be modified or improved. The dimensions of fibers created by electroprocessing are much finer than most other filter mesh components. Also, the porosity of the final product can be accurately determined depending on the many variable conditions of electroprocessing.

When electroprocessing a polymer matrix onto a substrate, the attachment of polymer fibers to the substrate must be considered. In an application where a fiber matrix is electroprocessed onto a filter comprising a fine wire mesh, the polymer does not automatically adhere or stick to the mesh. However, it is important that the fibers stay attached to the wire mesh (or other filter material). Delamination can reduce or prevent the effectiveness of the electroprocessed matrix. If the filter is implanted in vivo, delamination can have more serious ramifications.

### SUMMARY OF THE INVENTION

Accordingly, it is an object of the present invention to provide a solution to the potential problem of delamination. In the present invention, a fiber matrix is fused to a filter substrate.

In a first embodiment, a medical device filters a fluid in a lumen of a patient's body. That device includes a wire frame comprising a plurality of wires oriented to define a perimeter. It further includes a fiber matrix secured to that wire frame, the fiber matrix having fibers forming a boundary about each of a multiplicity of pores, the fiber matrix and the wire frame together forming a filter carried by a guide wire. The filter is collapsible prior to deployment and expandable to extend outward from the guide wire such that the filter engages a wall defining the lumen. The wire frame and fiber matrix are constructed and arranged to prevent passage of particulate matter while allowing passage of fluid through the pores. The fiber matrix is further fused to the wire frame. The fiber matrix may be heat fused, chemically fused, or mechanically bonded to the wire frame.

In another embodiment, a medical device filters fluid passing through a lumen in a patient's body. The device includes a flexible frame including a plurality of wires intersecting to define a perimeter of an open space. The device further includes an electrospun matrix including a multiplicity of fibers, the matrix fused to the frame and extending across the open space to define a multiplicity of pores. The fiber matrix may be heat fused to the wire frame, chemically fused to the wire frame, or fused by mechanical binding to the wire frame.

Still further, the invention includes a method of anchoring an electrospun polymer matrix to a filter substrate. The method includes providing a filter substrate, electrospinning a matrix of polymer fibers onto the filter substrate, and then fusing the matrix of polymer fibers onto the filter substrate. The step of fusing the polymer fibers onto the substrate may comprise heating at least a portion of the matrix to fuse it or it may comprise heating the entire matrix and substrate to fuse the matrix to the substrate. The matrix may also be pretreated with a chemical agent adapted to promote bonding of the matrix of polymer fibers to the filter substrate. The matrix and substrate may together be chemically treated to bond the matrix of polymer fibers to the substrate. Alternatively, the matrix of polymer fibers may be mechanically bonded onto the filter substrate to fuse it thereto.

### BRIEF DESCRIPTION OF THE DRAWINGS

FIGS. 1 and 2 are scanning electron micrographs of a matrix of electrospun nylon on a windsock type blood filter (magnification 15× and 120× respectively).

FIGS. 3 and 4 are scanning electron micrographs of an electrospun nylon matrix on a windsock type blood filter as shown in FIG. 1 (magnfication 950× and 190× respectively). These figures are of the open end of the filter that was heat-treated with a red hot scalpel blade to fuse the polymer fibers to the filter substrate.

FIGS. 5, 6 and 7 are scanning electron micrographs displaying heat bonding of a electrospun nylon matrix to a screen (magnification 22×, 180× and 650× respectively).

FIGS. 8, 9 and 10 display scanning electron micrographs showing the heat bonding of an electrospun nylon matrix to a windsock type blood filter (magnification 22×, 37×, 65× and 400× respectively).

### DETAILED DESCRIPTION

The solution to the problem of delamination of an electroprocessed matrix on a filter is to use one or more fusion techniques to anchor the electroprocessed matrix to the filter. The solutions include variations of heat fusion, chemical fusion and/or mechanical binding. The following discussion relates to detailed options and examples of anchoring an electrospun matrix of fibers to a filter. Specifically, a Microvena® blood filter, Trap 2 windsock design is used. The filters are made up of a mesh of twenty-four or forty-eight wires of a nickel/titanium alloy. The filter having twenty-four wires uses 0.002 inch diameter wire and has an average pore size of 215-220 microns. The filter having forty-eight wires uses 0.0015 inch diameter wire and has a maximum pore size of 253 microns.

Although described in connection with a windsock-type of blood filter, the invention is envisioned for use with any filters or other medical devices for filtering fluid in a lumen of a patient's body. The filter may be constructed of any material such as metal, plastic, ceramic, hybrids thereof, etc. In essence, the filter may be any material onto which a matrix may be electroprocessed. Typically, the filter is a wire frame and includes a plurality of wires oriented to define a perimeter. The fiber matrix is fused or otherwise secured onto this wire frame, with the fibers forming a boundary about each of a multiplicity of pores. The fiber matrix and the wire frame together form the filter.

In at least one embodiment, the filter is carried by a guidewire with the filter being collapsible prior to deployment, the filter being expandable to extend outward from the guidewire such that the filter engages a wall defining the lumen. The wire frame and fiber matrix are constructed and arranged to prevent passage of particulate matter while allowing passage of fluid through the pores. This and other types of frame/matrix filters are discussed in more detail in the published application referred to earlier and incorporated herein by reference—Publication No. US2002/0128680 A1, published Sep. 12, 2002.

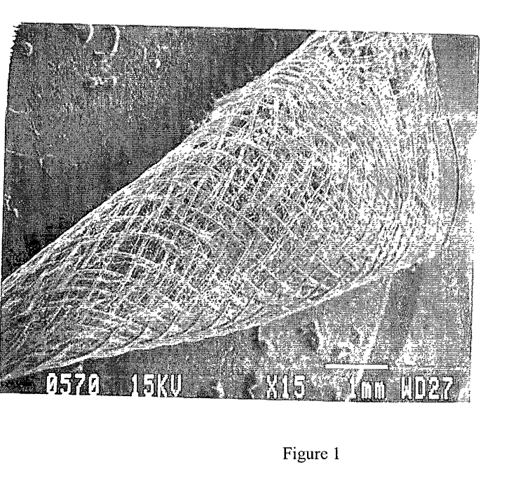

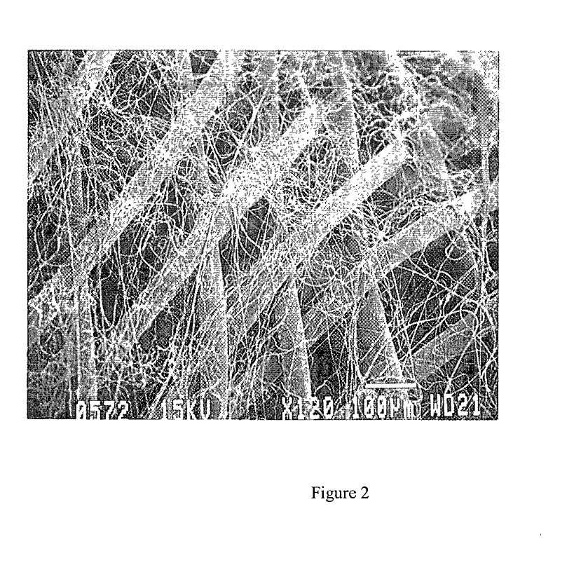

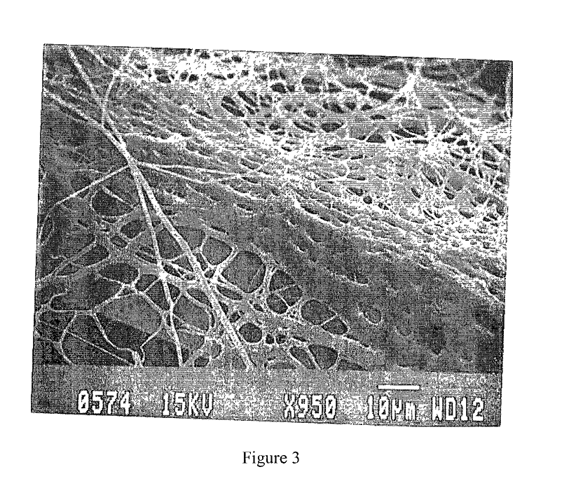

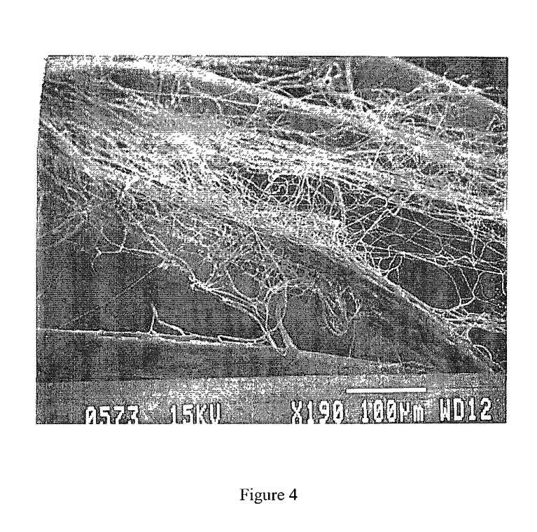

One option to prevent delamination of an electrospun polymer matrix from a filter frame is through the use of heat fusion. When electrospinning a polymer onto a Microvena® filter, the electrospun matrix can be easily removed from the filter. This easy removal (delamination) is presumably not acceptable for the intended use of the filter. Accordingly, an electrospun matrix of nylon from HFIP solution was formed onto a Microvena® filter. A red-hot scalpel blade was then used to melt the polymer covering the large opening of the filter after electrospinning. The result was the fusion of the polymer around the rim or large opening of the filter. FIGS. 1 and 2 display the filter having the electrospun matrix of fibers on it. FIGS. 3 and 4 show the portion of the matrix that was heat-treated with the hot blade to fuse the fibers to the filter.

A variation of this heat fusion solution is to apply heat to the entire filter that is coated with the polymer matrix. This type of comprehensive heat treatment can fuse the entire polymer matrix coating to the filter and not just the leading edge around the opening as noted earlier using the hot blade. Also, the filter can be heated before and/or during the electroprocessing step so that the fibers fuse to the hot filter substrate on contact. The temperatures used and the time of heat treatment will of course vary depending on the type of polymer matrix, the degree of fusion, the size of the overall filter, the thickness of the matrix, and many other processing conditions.

A further option for preventing delamination is to use chemical fusion techniques. The substrate may be pre-treated with a chemical agent to better bond the electroprocessed fibers when they are spun onto the substrate. Also, after the matrix is electroprocessed onto the substrate, the entire device may be coated or dipped into a solvent. The solvent may be any compound or combination of compounds that enhance the bond between the polymer matrix and the substrate, but one very convenient solvent is the solvent that may be used in the electrospinning process itself. This chemical fusion may be used universally as described in the dipping method, or it may be used in a more local fashion, for instance, around the opening of a filter. The processing conditions will vary greatly depending on the nature of the polymer matrix, the substrate material, the size of the area to be fused, the type and concentration of solvent, and many other processing features that may be important on a case by case basis.

A still further option for preventing delamination includes the mechanical binding of the matrix onto the substrate. For instance, a thread or other thick fiber may be sewn into the electroprocessed matrix and wrapped around and into the substrate. Further, in the example of the filter having a large opening, a metallic or polymer ring structure may be secured around the opening to press the matrix against the rim to prevent the leading edge of the electrospun matrix from delaminating. Again, the decision of whether to bind a portion or effectively all of the matrix to a substrate will depend on the application and specifications. The particular types of materials that are used to mechanically bind the matrix to the substrate will similarly vary depending on the application.

Finally, a combination of two or more of the foregoing methods may be used. Depending on the specifications on a case-by-case basis, it may be desirable or required to use multiple techniques to insure against delamination.

Another option that may incorporate one or more of the foregoing techniques is directed to electroprocessing variations. A polymer may be coated onto a substrate by electrospraying of polymer droplets. Polymer fibers may then be electrospun onto the coated substrate. In a variation, the coating step by electrospraying could be done after the polymer fibers are spun onto a substrate. The polymers used to electrospray a coating and electrospin a matrix may be the same or they may be different. For instance, the coating polymer may have a lower melt index so that the process of heat fusion will not affect the other polymer fibers. There could also be variations in solubility, for instance, so that chemical fusion could be carried out with minimal effect on electrospun fibers. Other electroprocessing variations could also be manipulated in combination with the other fusion techniques described herein to better anchor a polymer to a substrate.

Still further, the electroprocessed matrix could itself be modified in order to aid in the purpose of the filter. Either before, during or after the electroprocessing, the matrix (or matrix-forming material) can be chemically treated. For instance, heparin or another pharmaceutical agent may be bound to or incorporated into the matrix. The electroprocessed matrix itself could be a drug delivery device to assist in the patient treatment. A copending application discusses in detail some drug delivery options in electroprocessed matrices. That application has been published as Publication No. WO 02 32397 (PCT/US01/32301), filed Oct. 18, 2001, and is incorporated herein by reference.

### EXAMPLE

In an attempt to modify a Microvena® distal protection device with an average pore size just above 200 microns, nylon nanofibers were electrospun onto a standard window screen. The screen served as a model for testing this procedure since its material parameters are similar to the distal protection device (grid size, etc.). Nylon polymer (Rilsan (R) AMNO; Elf Atochem North America, Inc., Philadelphia, Pa.) was placed into 1,1,1,3,3,3-hexaflouroisopropanol (HFIP) overnight to dissolve. The solution was then electrospun onto a screen through an 18 gauge nozzle and the resultant composite was placed in an oven varied between 150-170° C. for set times. The screens were then removed from the oven and agitated by hand to test for proper bonding. Initially, the testing of various nylon/HFIP concentrations, mandrel to syringe tip distances (M-S), voltages, syringe pump flow rates, and oven exposure times and temperatures were deemed unsuccessful since the nylon would not stick to the screen.

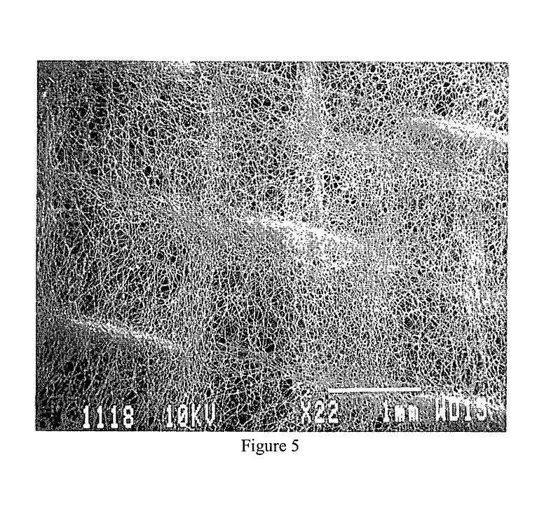

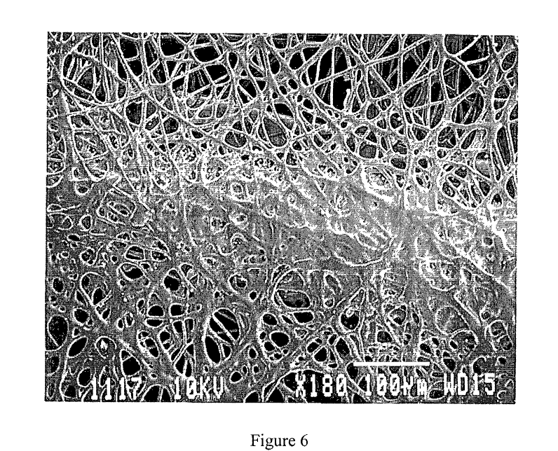

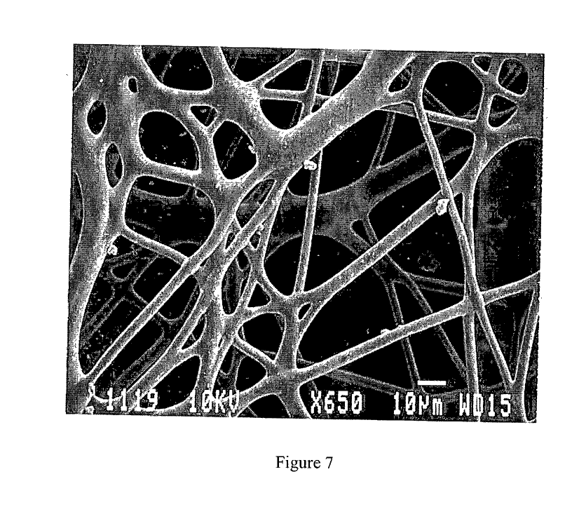

However, successful bonding of the electrospun nylon nanofibers to the screen was finally achieved by using a nylon/HFIP solution (169 mg/ml). A blunt ended 25-gauge needle was attached to the syringe. The syringe pump flow rate was then set at 10 ml/hr and the voltage was adjusted to 16 kV. After spinning the nylon onto the filter, the composite was placed in an oven (162±4° C.) for 110 seconds. The composite was then removed from the mandrel and articulated to ensure proper bonding. The nylon could not be peeled off the metal screen, and instead, the fibers remained attached. Investigation under scanning electron microscopy revealed that the nylon fibers appeared melted onto the metal screen at the points of nylon binding. In addition, fiber structure was retained across the spaces of potential filtration. These results are shown in FIGS. 5-7.

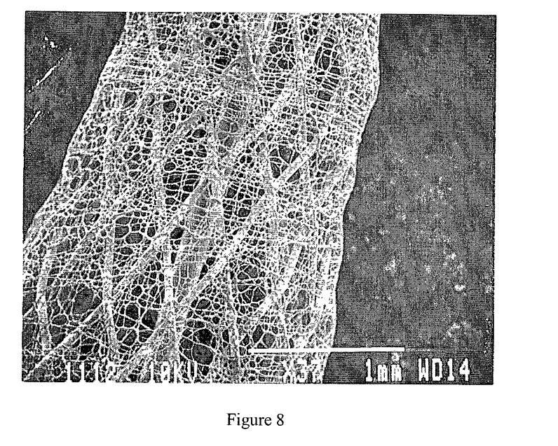

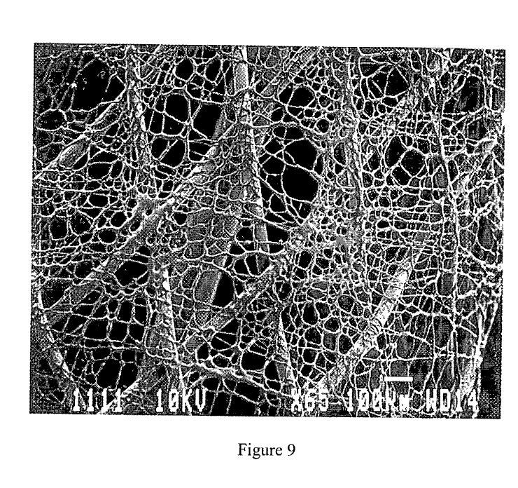

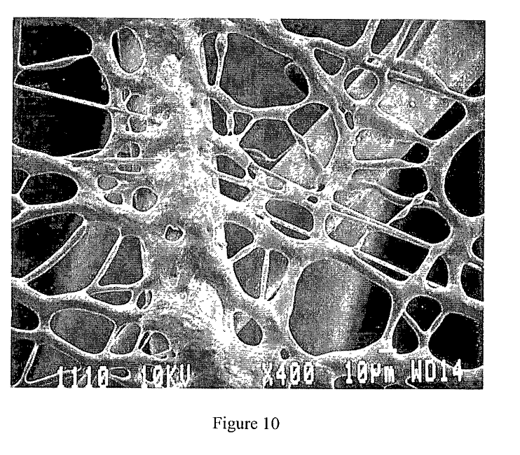

Finally, a nylon matrix as described herein was electrospun on an actual Microvena distal protection device made from Nitinol (NiTi). The same processing and heat fusion parameters as those described earlier were used herein. The results of this study are shown in FIGS. 8-10.

While the invention has been described with reference to specific embodiments thereof, it will understood that numerous variations, modifications and additional embodiments are possible, and accordingly, all such variations, modifications, and embodiments are to be regarded as being within the spirit and scope of the invention.

What is claimed is:

1. A medical device for filtering a fluid in a lumen of a patient's body, comprising:
a wire frame comprising a plurality of wires oriented to define a perimeter;
a fiber matrix secured to said wire frame, said fiber matrix having fibers forming a boundary about each of a multiplicity of pores, said fiber matrix and said wire frame together forming a filter carried by a guidewire with said filter being collapsible prior to deployment, said filter being expandable to extend outward from said guidewire such that said filter engages a wall defining said lumen, said wire frame and said fiber matrix being constructed and arranged to prevent passage of particulate matter while allowing passage of fluid through said pores; and
wherein the fiber matrix is fused to said wire frame.

2. A medical device as described in claim 1, wherein the fiber matrix is heat fused to said wire frame.

3. A medical device as described in claim 1, wherein the fiber matrix is chemically fused to said wire frame.

4. A medical device as described in claim 1, wherein the fiber matrix is fused to said wire frame by mechanical binding.

5. A medical device for filtering fluid passing through a lumen in a patient's body, comprising:
a flexible frame including a plurality of wires intersecting to define a perimeter of an open space; and
an electrospun matrix including a multiplicity of fibers, the matrix fused to the frame and extending across the open space to define a multiplicity of pores.

6. A medical device as described in claim 5, wherein the fiber matrix is heat fused to said wire frame.

7. A medical device as described in claim 5, wherein the fiber matrix is chemically fused to said wire frame.

8. A medical device as described in claim 5, wherein the fiber matrix is fused to said wire frame by mechanical binding.

9. A method of anchoring an electrospun polymer matrix to a filter substrate, comprising the steps of:
providing a filter substrate;
electrospinning a matrix of polymer fibers onto the filter substrate;
fusing the matrix of polymer fibers onto the filter substrate.

10. A method as described in claim 9, wherein the step of fusing the matrix of polymer fibers onto the filter substrate comprises heating at least a portion of the matrix to fuse it to the filter substrate.

11. A method as described in claim 9, wherein the entire matrix and substrate is heated to fuse the matrix to the filter substrate.

12. A method as described in claim 9, wherein the step of fusing the matrix of polymer fibers onto the filter substrate comprises pre-treating the filter substrate with a chemical agent adapted to promote bonding of the matrix of polymer fibers to the filter substrate.

13. A method as described in claim 9, wherein the step of fusing the matrix of polymer fibers onto the filter substrate comprising chemically treating the matrix and substrate to bond the matrix of polymer fibers to the filter substrate.

14. A method as described in claim 9, wherein the step of fusing the matrix of polymer fibers onto the filter substrate comprises mechanically binding the matrix onto the substrate.
